(19) United States
(12) Patent Application Publication
(10) Pub. No.: US 2008/0097271 A1
(43) Pub. Date: Apr. 24, 2008

(54) Devices and methods for the delivery of hemostatic agents to bleeding wounds

(75) Inventors: Denny Lo, Bethlehem, CT (US); Raymond Huey, Orange, CT (US)

Correspondence Address: MICHAUD-DUFFY GROUP LLP, 306 INDUSTRIAL PARK ROAD, SUITE 206, MIDDLETOWN, CT, 06457, US

(73) Assignee: Z-Medica Corporation, Wallingford, CT (US)

(21) Appl. No.: 11/584,079

(22) Filed: Oct. 20, 2006

(51) Int. Cl. A61F 13/40

(52) U.S. Cl. 602/48

(57) ABSTRACT

A hemostatic agent applicable to a bleeding wound to promote the clotting of blood comprises a first component and a second component, both components being in particle form and commingled with each other, and both components having hemostatic properties. A device incorporating such an agent comprises a receptacle for retaining the agent in particulate form therein. At least a portion of the receptacle is defined by a mesh having openings therein through which the blood may flow to come into contact with the particles of the hemostatic agent. A pad for controlling the flow of blood from a bleeding wound comprises a mesh structure and the hemostatic agent retained therein. A bandage applicable to a bleeding wound is defined by a substrate, a mesh mounted on the substrate, and the hemostatic agent retained in the mesh.

### TECHNICAL FIELD

The present invention relates generally to devices for promoting hemostasis and, more particularly, to hemostatic agents and devices incorporating such agents.

### BACKGROUND OF THE INVENTION

Blood is a liquid tissue that includes red cells, white cells, corpuscles, and platelets dispersed in a liquid phase. The liquid phase is plasma, which includes acids, lipids, solubilized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances, animals, including humans, can be wounded. Often bleeding is associated with such wounds. In some circumstances, the wound and the bleeding are minor, and normal blood clotting functions in addition to the application of simple first aid are all that is required. Unfortunately, however, in other circumstances substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stanch the flow of blood in a timely manner.

Moreover, severe wounds can often be inflicted in remote areas or in situations, such as on a battlefield, where adequate medical assistance is not immediately available. In these instances, it is important to stop bleeding, even in less severe wounds, long enough to allow the injured person or animal to receive medical attention.

In an effort to address the above-described problems, materials have been developed for controlling excessive bleeding in situations where conventional aid is unavailable or less than optimally effective. Although these materials have been shown to be somewhat successful, they are sometimes not effective enough for traumatic wounds and tend to be expensive. Furthermore, these materials are sometimes ineffective in some situations and can be difficult to apply as well as remove from a wound.

Additionally, or alternatively, the previously developed materials can produce undesirable side effects. For example, one type of prior art blood clotting material is generally a powder or a fine particulate in which the surface area of the material often produces an exothermic reaction upon the application of the material to blood. Oftentimes excess material is unnecessarily poured onto a wound, which can exacerbate the exothermic effects. Depending upon the specific attributes of the material, the resulting exothermia may be sufficient to cause discomfort to or even burn the patient. Although some prior art patents specifically recite the resulting exothermia as being a desirable feature that can provide clotting effects to the wound that are similar to cauterization, there exists the possibility that the tissue at and around the wound site may be undesirably impacted.

Some of the previously developed materials can also be difficult to apply and maintain in contact with the wound site. For example, some prior art blood clotting materials include mesoporous bioactive glasses and material composites in which a particle is composed of several layers of disparate materials. Depending upon the size of the particle and the location of the wound, delivery of particulate material may be difficult and result in waste, particularly if the particles are poured from a container and the exact location of the wound is not discerned or if the material is applied by a person other than the wounded person. Also, utilizing the material in these manners under stressful conditions (e.g., in low-visibility conditions) may contribute to the difficulty of the application.

Furthermore, to remove such materials from wounds, irrigation of the wound is often required. If an amount of material is administered that causes discomfort or burning, the wound may require immediate flushing. In instances where a wounded person or animal has not yet been transported to a facility capable of providing the needed irrigation, undesirable effects or over-treatment of the wound may result.

Bleeding can also be a problem during surgical procedures. Apart from suturing or stapling an incision or internally bleeding area, bleeding is often controlled using a sponge or other material used to exert pressure against the bleed site and/or absorb the blood. However, when the bleeding becomes excessive, these measures may not be sufficient to stop the flow of blood. Moreover, any highly exothermic bleed-control material may damage the tissue surrounding the bleed site and may not be configured for easy removal after use.

Based on the foregoing, it is a general object of the present invention to provide a hemostatic agent that overcomes or improves upon the drawbacks associated with the prior art. It is also a general object of the present invention to provide devices capable of applying such hemostatic agents.

### SUMMARY OF THE INVENTION

According to one aspect, the present invention resides in a hemostatic agent applicable to a bleeding wound to promote the clotting of blood when the agent is brought into contact with the wound. The hemostatic agent comprises a first component and a second component, both components being in particle form and commingled with each other, and both components having hemostatic properties.

According to another aspect, the present invention resides in a device for promoting the clotting of blood when applied to a bleeding wound. The device comprises a receptacle for retaining a hemostatic agent in particulate form therein, the hemostatic agent comprising a first component having hemostatic properties commingled with a second component having hemostatic properties. At least a portion of the receptacle is defined by a mesh having openings therein through which the blood may flow to come into contact with the particles of the hemostatic agent.

According to another aspect, the present invention resides in a pad for controlling the flow of blood from a bleeding wound. The pad comprises a mesh structure and a hemostatic agent retained therein. The mesh structure is defined by openings sized to accommodate the blood flow therethrough. The hemostatic agent comprises particles of a first component having hemostatic properties and particles of a second component having hemostatic properties, the particles of each component being commingled with the particles of the other component. The pad also includes a support attached to the mesh structure.

According to another aspect, the present invention resides in a bandage applicable to a bleeding wound. The bandage is defined by a substrate, a mesh mounted on the substrate, and a hemostatic agent retained in the mesh. The mesh is defined by a plurality of members arranged to define openings, the openings being dimensioned to accommodate blood flow therethrough when the bandage is applied to the bleeding wound. The hemostatic agent is defined by particles of a first component having hemostatic properties and particles of a second component having hemostatic properties, the particles of each being commingled to form a homogenous mixture.

In the preferred embodiments of the hemostatic agents and the devices disclosed herein, the first component may be, for example, a zeolite, and the second component may be, for example, a clay such as kaolin.

An advantage of the present invention is that the zeolite component in combination with the clay component causes less of an exothermic reaction with blood than if the zeolite was used alone. In particular, the presence of clay tempers the exothermic effects experienced at the wound site by causing a less aggressive drawing of moisture from the blood. It is theorized that the less aggressive drawing of moisture from the blood is the result of a less rapid transfer of moisture from the wound. However, the porous nature of the hemostatic agent still allows water to be wicked away to cause thickening of the blood, thereby facilitating the formation of clots.

Another advantage is that the hemostatic agent of the present invention reacts more exothermically with blood than does one that is all or substantially all clay material. A small amount of heat aids in the process of coagulating blood. Accordingly, by blending proportionate amounts of a component (e.g., zeolite) that produces an exothermic reaction with blood together with clay, the total amount of heat can be modulated and some amount of heat can be desirably generated to facilitate the clotting of the blood.

Another advantage is that the hemostatic properties of the hemostatic agent can be “tuned” depending on the needs at hand. This tuning can be easily effected by varying the ratio of the individual components in the agent. More particularly, the amount of zeolite relative to the clay can be adjusted to control the amount of heat generated at a wound site. Controlling the amount of heat at a wound site may be useful in the treatment of certain patients such as pediatric or geriatric patients or when the wound being treated is in a particularly sensitive or delicate area.

Still another advantage of the present invention is that the agents and devices of the present invention are easily applied to open wounds. Particularly when the hemostatic agent is retained in a mesh or similar device, the device can be readily removed from a sterilized packaging and placed or held directly at the points from which blood emanates to cause clotting.

### BRIEF DESCRIPTION OF THE DRAWINGS

FIG. 1 is a schematic representation of a blood clotting device of the present invention.

FIG. 2 is a side view of the blood clotting device of FIG. 1 illustrating the retaining of molecular sieve particles in a mesh container.

FIG. 3 is a side view of a pressure pad incorporating the molecular sieve particles encapsulated in a mesh container for pressure application to a bleeding wound.

FIG. 4 is a perspective view of a bandage incorporating the molecular sieve particles in a mesh container for application to a bleeding wound.

FIG. 5 is a graphical representation illustrating comparative in vitro clot times of blood.

### DETAILED DESCRIPTION OF THE PREFERRED EMBODIMENTS

Disclosed herein are hemostatic devices and hemostatic agents that are applicable to bleeding wounds to promote hemostasis. The hemostatic agents generally include two-component mixtures of particles having hemostatic qualities, such mixtures being contained within mesh bags, perforated containers, or similar devices that, when brought into contact with a bleeding wound, can minimize or stop blood flow by absorbing at least portions of the liquid phases of the blood, thereby facilitating clotting. The mixtures generally include particles of a molecular sieve material and particles of a clay material. The present invention is not limited to two-component mixtures, however, as other materials (e.g., anti-infective agents and the like) in particle form may be included as third or subsequent components.

In one preferred embodiment of the present invention, the molecular sieve material is a zeolite and the clay material is kaolin. The present invention is not limited in this regard, however, as other molecular sieve materials and other clays are within the scope of the present invention. Bioactive glasses, siliceous oxides, diatomaceous earth, and combinations thereof may also be used in place of (or in addition to) either or both the zeolite and the clay.

As used herein, the term “zeolite” refers to a crystalline form of aluminosilicate having one or more ionic species such as, for example, calcium and sodium moieties and the ability to be dehydrated without experiencing significant changes in the crystalline structure. Typically, the zeolite is a friable material that includes oxides of calcium, sodium, aluminum, and silicon in addition to water. The calcium portion contains crystals that are about 5 angstroms in size, and the sodium portion contains crystals that are about 4 angstroms in size. The preferred molecular structure of the zeolite is an “A-type” crystal, namely, one having a cubic crystalline structure that defines round or substantially round openings. One preferred zeolite is that designated as “5A,” which indicates a crystal size of about 5 angstroms and having a cubic crystalline structure defining round or substantially round openings.

The zeolites may be mixed with or otherwise used in conjunction with other materials having the ability to be dehydrated without significant changes in crystalline structure. Such materials include, but are not limited to, magnesium sulfate, sodium metaphosphate, calcium chloride, dextrin, combinations of the foregoing materials, and hydrates of the foregoing materials.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, analcite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides.

Various materials may be mixed with, associated with, or incorporated into the zeolites to maintain an antiseptic environment at the wound site or to provide functions that are supplemental to the clotting functions of the zeolites. Exemplary materials that can be used include, but are not limited to, pharmaceutically-active compositions such as antibiotics, antifungal agents, anti-infective agents, antimicrobial agents, anti-inflammatory agents, analgesics, antihistamines (e.g., cimetidine, chloropheniramine maleate, diphenhydramine hydrochloride, and promethazine hydrochloride), compounds containing silver ions, and the like. Other materials that can be incorporated to provide additional hemostatic functions include ascorbic acid, tranexamic acid, rutin, and thrombin. Botanical agents having desirable effects on the wound site may also be added. Any of the foregoing materials, individually or in combination, may also be present in particle form and blended with the particles of clay and/or the zeolite particles.

For use in the present invention, the zeolite is preferably in particle form. As used herein, “particles” include beads, pellets, granules, rods, or any other surface morphology or combination of surface morphologies. Irrespective of the surface morphology, the zeolite particles are about 0.2 mm (millimeters) to about 10 mm, preferably about 0.5 mm to about 5 mm, and more preferably about 1 mm to about 2 mm in effective diameter. The effective diameter is the average length of a plurality of imaginary straight lines drawn through the geometric center of a particle.

The particle form of the zeolite may be obtained by any suitable operation. For example, particilized zeolite may be obtained from powder stock by any suitable method such as extrusion, pelletizing, or the like. Particlized zeolite may also be obtained by rolling, pulverizing, or otherwise crushing larger chunks of zeolite. The present invention is not limited in this regard, however, as other methods of manipulating the zeolite into particle form are within the scope of the present invention.

As used herein, the term “clay” refers to a crystalline form of hydrated aluminum silicate. The crystals of clay are irregularly shaped and insoluble in water. The combination of some types of clay with water may produce a mass having some degree of plasticity. Depending upon the type of clay, the combination thereof with water may produce a colloidal gel having thixotropic properties.

The clay utilized in the hemostatic agents and devices of the present invention is preferably kaolin, which is an aluminum phyllosilicate comprising about 50% alumina, about 50% silica, and trace impurities. Because of its high purity, kaolin has a high fusion point and is the most refractory of all clays, thus making it suitable for ceramic compositions, refractory processes, catalytic processes, and other industrial uses as well as in cosmetics, pharmaceuticals, and water treatment systems.

More preferably, the clay is Edgar's plastic kaolin (hereinafter “EPK”), which is a water-washed kaolin clay that is mined and processed in and near Edgar, Fla. Edgar's plastic kaolin has desirable plasticity characteristics, is castable, and when mixed with water produces a thixotropic slurry. Other clays such as attapulgite or bentonite are also within the scope of the present invention and can be used individually, in combination with each other, or in combination with kaolin.

The EPK used in the present invention is particlized, dried, and fired to about 600 degrees C. In order to achieve a suitably homogenous mixture of the EPK to form the particles, a relatively high shear is applied to a mass of the EPK using a suitable mixing apparatus. Prior to shearing, the water content of the clay is measured and adjusted to be about 20% by weight to give a sufficiently workable mixture for extrusion and subsequent handling.

During the firing of the EPK, the material is vitrified. Vitrification is effected via repeated melting and cooling cycles to allow the EPK (or other clay material) to be converted into a glassy substance. With increasing numbers of cycles, the crystalline structure is broken down to result in an amorphous composition. The amorphous nature of the EPK allows it to maintain its structural integrity when subsequently wetted. As a result, the EPK maintains its structural integrity when wetted during use, for example, when applied to blood.

As with the zeolite, the particles of clay may be beads, pellets, granules, rods, or any other surface morphology or combination of surface morphologies. Irrespective of the surface morphology, the clay particles are about 0.2 mm to about 10 mm, preferably about 0.5 mm to about 5 mm, and more preferably about 1 mm to about 2 mm in effective diameter.

The clay particles can be produced by any of several various methods. Such methods include mixing, extrusion, spheronizing, and the like. Equipment that can be utilized for the mixing, extruding, or spheronizing of the clay is available from Caleva Process Solutions Ltd. in Dorset, United Kingdom. Other methods include the use of a fluid bed or a pelletizing apparatus. Fluid beds for the production of clay particles are available from Glatt Air Technologies in Ramsey, N.J. Disk pelletizers for the production of clay particles are available from Feeco International, Inc., in Green Bay, Wis. Preferably, the clay is extruded through a suitable pelletizing device. The present invention is not limited in this regard, however, as other devices and methods for producing particlized clay are within the scope of the present invention.

In formulating the hemostatic agent for use with a hemostatic device, the particles of zeolite are blended and commingled with the particles of clay. The sizes of the particles for each of the zeolite and the clay are similar. Furthermore, the amounts of zeolite particles and clay particles is comparable. Such amounts may be determined on a weight basis or a volume basis. The present invention is not limited in this regard, however, as the particle sizes of each component may be dissimilar and/or the amounts of each component may be disparate. Variation in the particle sizes and/or the amounts of each component allows any heat generated from the application of the hemostatic agent to a bleeding wound to be modulated as desired.

It is believed that the cellular clotting mechanisms of both molecular sieve material and clay activate certain contact factors when applied to blood. More specifically, it is believed that zeolite and kaolin (particularly EPK) are different but complementary. While each material exhibits hemostatic qualities on its own, it is likely that the differences in the molecular structures of each initiate different mechanisms by which water in blood is absorbed to facilitate clotting functions.

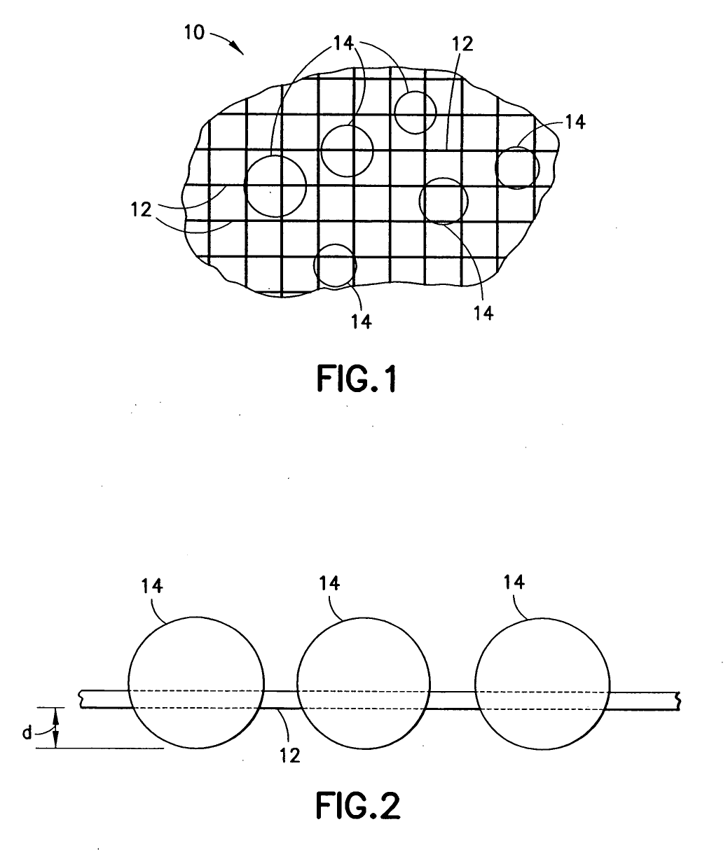

Referring now to FIG. 1, a hemostatic device into which the hemostatic agent is incorporated is shown. The device is a permeable pouch that allows liquid to enter to contact the hemostatic agent retained therein. Sealed packaging (not shown) provides a sterile environment for storing the hemostatic device until it can be used. The device, which is shown generally at 10 and is hereinafter referred to as “pouch 10,” comprises a screen or mesh 12 and the hemostatic agent 14 retained therein by the screen or mesh. The mesh 12 is closed on all sides and defines openings that are capable of retaining the hemostatic agent 14 therein while allowing liquid to flow through. As illustrated, the mesh 12 is shown as being flattened out, and only a few particles of hemostatic agent 14 are shown. The hemostatic agent 14 is a blend of zeolite particles and clay particles, the particles of each component being commingled to form a homogenous mixture.

The mesh 12 is defined by interconnected strands, filaments, or strips of material. The strands, filaments, or strips can be interconnected in any one or a combination of manners including, but not limited to, being woven into a gauze, intertwined, integrally-formed, and the like. Preferably, the interconnection is such that the mesh can flex while substantially maintaining the dimensions of the openings defined thereby. The material from which the strands, filaments or strips are fabricated may be a polymer (e.g., nylon, polyethylene, polypropylene, polyester, or the like), metal, fiberglass, or an organic substance (e.g., cotton, wool, silk, or the like).

Referring now to FIG. 2, the openings defined by the mesh 12 are dimensioned to retain the hemostatic agent 14 but to accommodate the flow of blood therethrough. Because the mesh 12 may be pulled tight around the hemostatic agent 14, the particles may extend through the openings by a distance d. If the particles extend through the openings, they are able to directly contact tissue to which the pouch 10 is applied. Thus, blood emanating from the tissue immediately contacts the hemostatic agent 14, and the water phase thereof is wicked into the zeolite and clay materials, thereby facilitating the clotting of the blood. However, it is not a requirement of the present invention that the particles protrude through the mesh.

To apply the pouch 10 to a bleeding wound, the pouch is removed from the packaging and placed on the bleeding wound. The hemostatic agent 14 in the mesh 12 contacts the tissue of the wound and/or the blood, and at least a portion of the liquid phase of the blood is adsorbed by the zeolite material, thereby promoting the clotting of the blood.

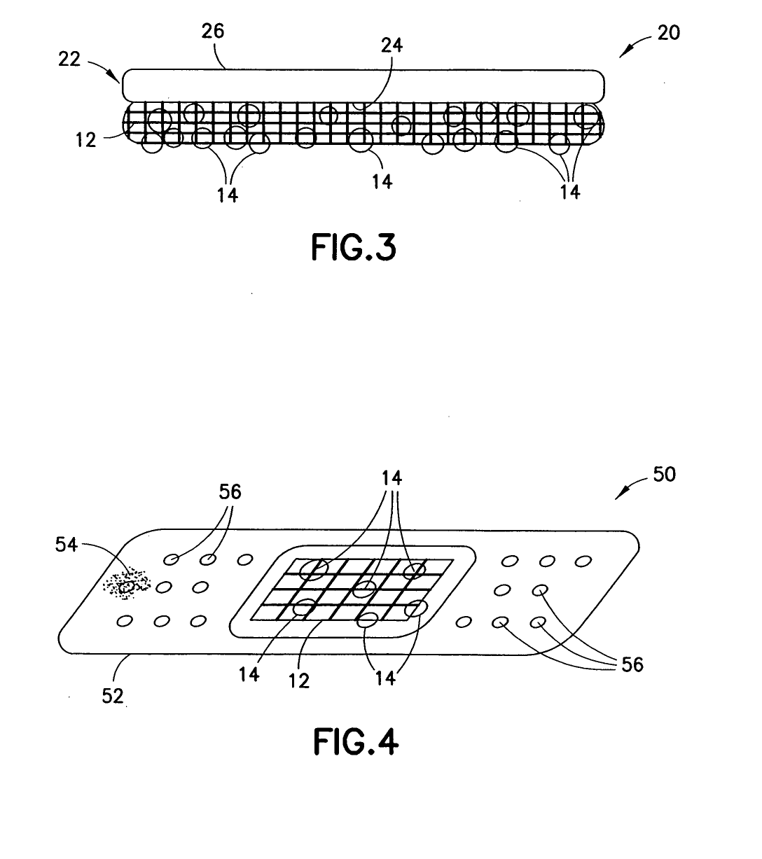

Another embodiment of the present invention is a pad which is shown at 20 with reference to FIG. 3 and is hereinafter referred to as “pad 20.” The pad 20 comprises the mesh 12, hemostatic agent 14 retained therein by the mesh 12, and a support 22 to which pressure may be applied in the application of the pad 20 to a bleeding wound. The mesh 12, as above, has openings that are capable of retaining the particles therein while allowing the flow of blood therethrough.

The mesh 12 is stitched, glued, clamped, or otherwise mounted to the support 22. The support 22 comprises an undersurface 24 against which the hemostatic agent 14 is held by the container 12 and a top surface 26. The undersurface 24 is impermeable to the hemostatic agent 14 (migration of the particles into the support 22 is prevented) and is further resistant to the absorption of water or other fluids. The top surface 26 is capable of having a pressure exerted thereon by a person applying the pad 20 to a bleeding wound or by a weight supported on the top surface 26. The entire support 22 is rigid or semi-rigid so as to allow the application of pressure while minimizing discomfort to the patient.

To apply the pad 20 to a bleeding wound, the pad 20 is removed from its packaging and placed on the bleeding wound. As with the pouch of the embodiment of FIGS. 1 and 2, the hemostatic agent 14 is either in direct contact with the tissue of the wound or are in direct contact with the blood. Pressure may be applied to the wound by pressing on the top surface 26 with a hand or by placing a weight on the surface, thereby facilitating the contact between the particles and the wound and promoting the adsorption of the liquid phase of the blood. The pad 20 (with or without a weight) may also be held onto the wound using a strapping device such as a belt, an elastic device, hook-and-loop material, combinations of the foregoing devices and materials, and the like.

Referring now to FIG. 4, another embodiment of the present invention is a bandage, shown at 50, which comprises particles of the hemostatic agent 14 retained in a mesh 12 and mounted to a flexible substrate 52 that can be applied to a wound (for example, using a pressure-sensitive adhesive to adhere the bandage 50 to the skin of a wearer). The mesh 12 is stitched, glued, or otherwise mounted to a substrate 52 to form the bandage 50.

The substrate 52 is a plastic or a cloth member that is conducive to being retained on the skin of an injured person or animal on or proximate a bleeding wound. An adhesive 54 is disposed on a surface of the substrate 52 that engages the skin of the injured person or animal. Particularly if the substrate 52 is a non-breathable plastic material, the substrate may include holes 56 to allow for the dissipation of moisture evaporating from the skin surface.

### EXAMPLE 1

### Comparison of Clot Times of Zeolite

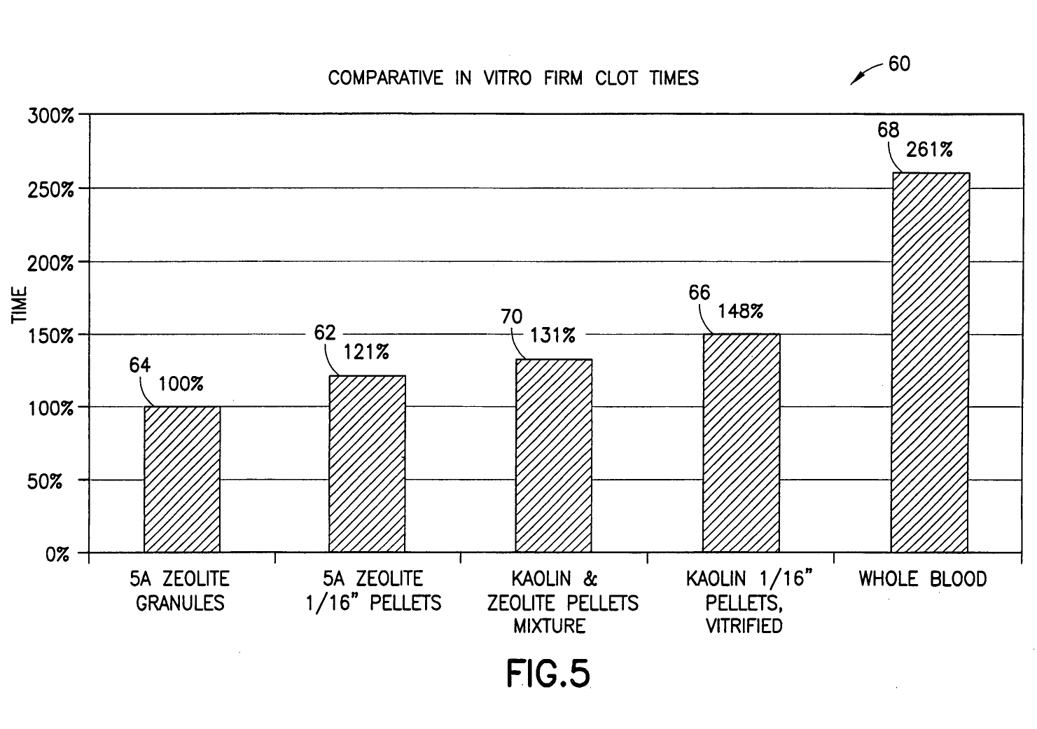

The in vitro clot times of 5A zeolite granules and of 5A zeolite pellets was measured. The granules had an average particle size of about 0.7 mm (0.3 mm to about 1.0 mm), and the pellets had an average effective diameter of about 1.6 mm ( 1/16 inch). Referring to FIG. 5, a graphical representation of the comparison of in vitro clot times is shown at 60. The pellets showed clot times 62 that were about 21% longer than the clot times 64 of the granules.

### EXAMPLE 2

### Comparison of Clot Times of Various Hemostatic Materials

Referring again to FIG. 5, the in vitro clot times 66 of kaolin (EPK) clay pellets was measured and compared to the clot times 64 for 5A zeolite granules. To form the clay pellets, the EPK was extruded, dried, and fired. In a first trial, the extruded EPK pellets were dried to 300 degrees C. In a second trial, the extruded EPK pellets were dried to 600 degrees C. Firing the pellets to 600 degrees C. vitrified the EPK, thereby allowing the pellets to remain structurally intact when wetted with blood. The pellets fired to 600 degrees C. (the second trial) exhibited an average clot time that was 48% longer than the clot time 64 of 5A zeolite granules having an average particle size of about 0.7 mm (0.3 mm to about 1.0 mm) and 56.7% of the time 68 to clot whole blood without a hemostatic agent. A clot time 70 of a mixture of kaolin clay pellets and zeolite pellets was 31% longer than the clot time 64 for 5A zeolite granules. The peak in vitro adsorption temperatures for the zeolite granules, zeolite pellets, and kaolin/zeolite pellet mixtures was 74, 77, and 36 degrees C., respectively.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A hemostatic agent, comprising:
a first component in particle form, said first component having hemostatic properties; and
a second component in particle form commingled with said first component, said second component having hemostatic properties;
wherein when using said hemostatic agent to treat a bleeding wound, contacting said bleeding wound with said hemostatic agent causes blood flowing from said bleeding wound to clot.

2. The hemostatic agent of claim 1, wherein said first component is a zeolite.

3. The hemostatic agent of claim 1, wherein said second component is a clay.

4. The hemostatic agent of claim 3, wherein said clay is kaolin.

5. The hemostatic agent of claim 4, wherein said kaolin is Edgar's plastic kaolin.

6. The hemostatic agent of claim 3, wherein said clay is selected from the group consisting of bentonite, attapulgite, kaolin, and combinations of the foregoing.

7. The hemostatic agent of claim 1, wherein a specific amount of said first component is combined with a specific amount of said second component to modulate an exotherm generated by an application of said hemostatic agent to blood.

8. The hemostatic agent of claim 1, wherein the particle forms of said first component and said second component include beads, rods, granules, and irregular surface morphologies.

9. The hemostatic agent of claim 1, wherein particle sizes of said first component and said second component are about 0.2 mm to about 10 mm in effective diameter.

10. The hemostatic agent of claim 1, wherein particle sizes of said first component and said second component are about 0.5 mm to about 5 mm in effective diameter.

11. The hemostatic agent of claim 1, wherein particle sizes of said first component and said second component are about 1 mm to about 2 mm in effective diameter.

12. The hemostatic agent of claim 1, further comprising at least one additional component in particle form, said at least one additional component having a property that is at least one of wound healing, antibiotic, antifungal, anti-infective, antimicrobial, anti-inflammatory, analgesic, antihistamine, and hemostatic.

13. The hemostatic agent of claim 1, wherein at least one of said first component and said second component is selected from the group consisting of bioactive glasses, siliceous oxides, diatomaceous earth, and combinations of the foregoing.

14. A device for promoting the clotting of blood, comprising:
a receptacle for retaining a hemostatic agent in particle form therein, said hemostatic agent comprising a first component in particle form and having hemostatic properties commingled with a second component in particle form and having hemostatic properties, at least a portion of said receptacle being defined by a mesh having openings therein;
wherein when treating a bleeding wound, application of said device causes at least a portion of at least one of said first component and said second component to come into contact with blood through said openings.

15. The device for promoting the clotting of blood of claim 14, wherein said first component is a zeolite.

16. The device for promoting the clotting of blood of claim 14, wherein said second component is a clay.

17. The device for promoting the clotting of blood of claim 14, wherein said mesh structure is flexible.

18. The device for promoting the clotting of blood of claim 14, wherein at least one particle of said hemostatic agent protrudes through one of said openings.

19. The device for promoting the clotting of blood of claim 14, wherein effective diameters of the particles of said first component and said second component are about 0.2 mm to about 10 mm.

20. The device for promoting the clotting of blood of claim 14, wherein effective diameters of the particles of said first component and said second component are about 0.5 mm to about 5 mm.

21. The device for promoting the clotting of blood of claim 14, wherein effective diameters of the particles of said first component and said second component are about 1 mm to about 2 mm.

22. The device for promoting the clotting of blood of claim 16, wherein said clay is kaolin.

23. The device for promoting the clotting of blood of claim 22, wherein said kaolin is Edgar's plastic kaolin.

24. The device for promoting the clotting of blood of claim 16, wherein said clay is selected from the group consisting of bentonite, attapulgite, kaolin, and combinations of the foregoing.

25. The device for promoting the clotting of blood of claim 14, wherein a specific amount of said first component is combined with a specific amount of said second component to modulate an exotherm generated by an application of said device to said bleeding wound.

26. The device for promoting the clotting of blood of claim 14, wherein the particle forms of said first component and said second component include beads, rods, granules, and irregular surface morphologies.

27. The device for promoting the clotting of blood of claim 14, further comprising at least one additional component in particle form, said at least one additional component having a property that is at least one of wound healing, antibiotic, antifungal, anti-infective, antimicrobial, anti-inflammatory, analgesic, antihistamine, and hemostatic.

28. The device for promoting the clotting of blood of claim 14, wherein at least one of said first component and said second component is selected from the group consisting of bioactive glasses, siliceous oxides, diatomaceous earth, and combinations of the foregoing.

29. A pad for controlling bleeding, said pad comprising:
a mesh structure;
a hemostatic agent retained in said mesh structure, said hemostatic agent comprising particles of a first component having hemostatic properties and particles of a second component having hemostatic properties, said particles of said second component being commingled with said particles of said first component; and
a support attached to said mesh structure;
wherein said mesh structure is defined by openings sized to accommodate the flow of blood therethrough.

30. The pad of claim 29, wherein said first component is a zeolite and wherein said second component is a clay.

31. The pad of claim 29, wherein said particles of said first component and said particles of said second component each have effective diameters of about 0.2 mm to about 10 mm.

32. The pad of claim 29, wherein said particles of said first component and said particles of said second component each have effective diameters of about 0.5 mm to about 5 mm.

33. The pad of claim 29, wherein said particles of said first component and said particles of said second component each have effective diameters of about 1 mm to about 2 mm.

34. The pad of claim 29 wherein said support is configured to have a pressure applied thereto to enable said pad to be retained on a bleeding wound.

35. The pad of claim 30, wherein said clay is kaolin.

36. The pad of claim 35, wherein said kaolin is Edgar's plastic kaolin.

37. The pad of claim 30, wherein said clay is selected from the group consisting of bentonite, attapulgite, kaolin, and combinations of the foregoing.

38. The pad of claim 29, wherein a specific amount of said first component is combined with a specific amount of said second component to modulate an exotherm generated by an application of said pad to blood.

39. The pad of claim 29, wherein the particle forms of said first component and said second component include beads, rods, granules, and irregular surface morphologies.

40. The pad of claim 29, further comprising at least at least one additional component in particle form, said at least one additional component having a property that is at least one of wound healing, antibiotic, antifungal, anti-infective, antimicrobial, anti-inflammatory, analgesic, antihistamine, and hemostatic.

41. The pad of claim 29, wherein at least one of said first component and said second component is selected from the group consisting of bioactive glasses, siliceous oxides, diatomaceous earth, and combinations of the foregoing.

42. A bandage applicable to a bleeding wound, said bandage comprising:
a substrate;
a mesh mounted on said substrate; and
a hemostatic agent retained in said mesh, said hemostatic agent comprising particles of a first component having hemostatic properties and particles of a second component having hemostatic properties;
said mesh defined by a plurality of members arranged to define openings, said openings being dimensioned to accommodate the flow of blood therethrough.

43. The bandage of claim 42, further comprising an adhesive on said substrate, said adhesive being configured to facilitate the retaining of said bandage on the skin of a wearer.

44. The bandage of claim 42, wherein said first component is a zeolite and said second component is a clay.

45. The bandage of claim 44, wherein said clay is kaolin.

46. The bandage of claim 45, wherein said kaolin is Edgar's plastic kaolin.

47. The bandage of claim 44, wherein said clay is selected from the group consisting of bentonite, attapulgite, kaolin, and combinations of the foregoing.

48. The bandage of claim 42, wherein a specific amount of said first component is combined with a specific amount of said second component to modulate an exotherm generated by an application of said bandage to blood.

49. The bandage of claim 42, wherein the particle forms of said first component and said second component include beads, rods, granules, and irregular surface morphologies.

50. The bandage of claim 42, wherein said particles of said first component and said particles of said second component each have effective diameters of about 0.2 mm to about 10 mm.

51. The bandage of claim 42, wherein said particles of said first component and said particles of said second component each have effective diameters of about 0.5 mm to about 5 mm.

52. The bandage of claim 42, wherein said particles of said first component and said particles of said second component each have effective diameters of about 1 mm to about 2 mm.

53. The bandage of claim 42, further comprising at least at least one additional component in particle form, said at least one additional component having a property that is at least one of wound healing, antibiotic, antifungal, anti-infective, antimicrobial, anti-inflammatory, analgesic, antihistamine, and hemostatic.

54. The bandage of claim 42, wherein at least one of said first component and said second component is selected from the group consisting of bioactive glasses, siliceous oxides, diatomaceous earth, and combinations of the foregoing.
